(19) United States
(12) Patent Application Publication
(10) Pub. No.: US 2023/0149659 A1
(43) Pub. Date: May 18, 2023

(54) MULTIFUNCTIONAL AROMATHERAPY MACHINE

(71) Applicant: Shanghai Sunpai Trading Limited, Shanghai (CN)

(72) Inventor: Jianchao Xu, Shanghai (CN)

(21) Appl. No.: 17/525,937

(22) Filed: Nov. 14, 2021

(51) Int. Cl. A61M 21/02; F21V 23/00; F21V 33/00; A61M 11/00; A61N 5/06

(52) U.S. Cl. CPC A61M 21/02; F21V 23/003; F21V 33/0004; A61M 11/006; A61N 5/0618; F21Y 2115/10

(57) ABSTRACT

The present invention provides a multifunctional aromatherapy machine, including a water tank and a pedestal, wherein an aluminum ring is arranged between the water tank and the pedestal, and a circuit board, an atomizing sheet and an atomizing sheet pressing plate are arranged on the pedestal; the circuit board includes a control circuit, a power interface, a timing indicator lamp and a foglight knob are arranged on an outer side wall of the pedestal respectively, and a chassis induces the power interface. The multifunctional aromatherapy machine provided by the present invention has the characteristics of diversified functions, capability of controlling a plurality of functions with one key, convenience in use, low cost, convenience in maintenance and the like.

### FIELD OF TECHNOLOGY

The present invention relates to field of a humidifying and atomizing apparatus, and in particular, to a multifunctional aromatherapy machine.

### BACKGROUND

With the improvement of people's living standard, small applicants such as aromatherapy machines and humidifiers have entered thousands of households. The working principles of the aromatherapy machines and humidifiers are the same, all of which decompose or volatilize the added liquid substances to achieve the functions of performing air, improving air quality and increasing air humidity. If different liquid is selected, for example, aromatherapy essential oil is added, the beauty treatment, health care and conditioning effects can be achieved, memory can be enhanced, insomnia, anxiety and headache can be improved, and mood can be mediated.

The aromatherapy machine decomposes water molecules and dissolved plant essential oil into nanoscale cold fog with a diameter of 0.1-5 microns to be dispersed into the surrounding air through high-frequency electronic concussion generated by an ultrasonic concussion device, so that the air is full of various fragrances. However, the existing aromatherapy machine has few functions and is inconvenient to use. For example, in the prior art, the aromatherapy machine has numerous keys, each function is controlled by a key, and various functions cannot be controlled through one-key on and off; in addition, the existing aromatherapy machine only has the lighting function, cannot adjust light and brightness and has an impact on human sleep, so that the requirements of customers cannot be met.

Chinese patent application number: 201720359539.5, application date: Apr. 7, 2017, publication date: Sep. 7, 2018, and patent name: An aromatherapy machine. The patent discloses an aromatherapy machine, including a shell, wherein a first mounting hole is formed at the top of the shell, and an air inlet groove is formed at the bottom of the shell; a water tank is fixedly arranged in the shell, and a peripheral wall of the water tank is recessed inwards to form a first air inlet cavity; an upper end of the water tank is connected to a conical water tank cover, the top of the water tank cover is open, a maximum diameter of the water tank cover is greater than an outer diameter of the water tank, and a peripheral wall of the water tank cover is recessed inwards to form a second air inlet cavity; a middle cover is fixedly arranged in the shell and is arranged outside the water tank cover and the water tank, an upper end of the middle cover is upwards mounted in the first mounting hole and is open, the opening at the upper end of the middle cover communicates with the opening at the upper end of the water tank cover, an upper part of an inner wall of the middle cover is closely attached to the top of the water tank cover, and a middle part of the inner wall of the middle cover and a peripheral wall of the water tank cover are clamped through a buckle; an atomizing sheet is arranged on a bottom surface of the water tank, and an upper surface of the atomizing sheet is attached to a bottom surface of the water tank, and the aromatherapy machine has high atomization efficiency and can change indoor odor quickly and effectively.

The aromatherapy machine disclosed in the patent documents has a complex function and few functions, cannot be controlled by one key, is inconvenient to use and has a bad user experience.

### SUMMARY

An objective of the present invention is to provide a multifunctional aromatherapy machine. The aromatherapy machine only needs a foglight knob to control a fog outlet mode and realize timing setting and light setting, and is convenient to use and maintain.

To achieve the above objective, the present invention provides the following technical solution:

a multifunctional aromatherapy machine includes a pedestal, a water tank and a cover body, wherein the water tank includes a through hole formed in a bottom surface of the water tank and communicating with the pedestal, and an air outlet formed on an inner side wall of the water tank and communicating with the pedestal; a chassis is fixedly connected below the pedestal, the pedestal and the chassis form a built-in space with an opening above, and a fan for outputting flowing wind to the air outlet is arranged in the pedestal; a circuit board, an atomizing sheet and an atomizing sheet pressing plate are arranged in the pedestal, and the atomizing sheet is hermetically and fixedly connected to the through hole formed in the bottom surface of the water tank through the atomizing sheet pressing plate; the circuit board is arranged above the fan and is fixedly connected to the outer bottom surface of the water tank; a power interface, a timing indicator lamp, a key switch and a foglight knob are arranged on an outer side wall of the pedestal respectively; the power interface, the timing indicator lamp and the key switch are electrically connected to the circuit board respectively; an aluminum ring is arranged on an inner edge of an opening above the pedestal, and the water tank is fixedly connected to the pedestal through the aluminum ring; and the cover body includes a water tank cover arranged at the opening above the water tank, a lampshade arranged on the edge of the opening above the water tank, and an air outlet cover arranged in the water tank and covering the air outlet.

Further, the circuit board includes a control circuit. The control circuit includes the following circuits:

a power circuit, the power circuit being configured to control or transmit electric energy;

a boosting circuit, the boosting circuit being configured to boost a power signal;

a comparison circuit, the comparison circuit being configured to convert an analog signal into a digital signal;

an MCU main control module, the MCU main control module being configured to control and process circuit information;

a key circuit, the key circuit being configured to control the key switch;

a lamplight control circuit, the lamplight control circuit being configured to control lamplight intensity or color;

an atomization control circuit, the atomization control circuit being configured to control the atomizing sheet to perform atomization; and

a fan driving circuit, the fan driving circuit being configured to drive the fan to rotate,

the power circuit inputting the power signal to the MCU main control module through the boosting circuit and the comparison circuit, the MCU main control module controlling the lamplight control circuit and the atomization control circuit respectively through the key circuit, and the MCU main control module controlling the fan driving circuit.

Further, the water tank cover is provided with a fog outlet communicating with an interior of the water tank, a water retaining sheet is arranged at a port of the fog outlet communicating with the interior of the water tank, and the water retaining sheet is fixedly connected to an inside surface of the water tank cover.

Further, a fog outlet hole is formed at the top of the lampshade, and a size and a shape of the fog outlet hole of the lampshade correspond to those of the fog outlet of the water tank cover.

Further, a sealing ring is arranged between the water tank and the pedestal, and the sealing ring is sleeved on the aluminum ring and an upper surface of an outer edge of the pedestal.

Further, the MCU main control module includes a water shortage detection circuit, and the water shortage detection circuit is configured to detect the water amount of the water tank.

Further, the atomization control circuit includes a fog out detection circuit, and the fog out detection circuit is configured to detect the atomization amount.

Further, the atomization control circuit includes a timing circuit, and the timing circuit is configured to control the fog out time regularly.

Further, the lamplight control circuit includes an LED foglight, and the LED foglight includes a sleeping lamp and an atmosphere lamp.

Further, the key circuit includes a gear control circuit, and the gear control circuit includes a non-intermittent fog outlet mode gear control circuit and an intermittent fog outlet mode gear control circuit.

Further, a key base is arranged in a side wall of the pedestal, the foglight knob is inserted into the key base, and the key base is electrically connected to the key switch.

Further, a gray rubber mat adheres to the lower surface of the chassis and has an anti-skid effect on the bottom of the aromatherapy machine.

The technical solution provided by the present invention has the following beneficial effects:

(1) the aromatherapy machine disclosed by the present invention has exquisite overall design, high intelligent degree and diversified functions, and achieves the purpose of using a plurality of functions by controlling the key circuit with one key through the MCU main control module, that is, the present invention not only can regulate and control different lamplight modes through one foglight button, but also can control various different fog outlet function modes, has a timing function, is more convenient to use and has a strong user experience.

(2) The present invention has diversified functions, is convenient to disassemble, simple and convenient to maintain and low in maintenance cost in later period, and greatly prolongs the service life.

(3) The present invention has high intelligent degree, is an upgrade of the technology in the field of the aromatherapy machine, and is safer and reliable to use and suitable for universal popularization.

### BRIEF DESCRIPTION OF THE DRAWINGS

The accompany drawings constituting a part of the present application provide further understanding of the present invention. The schematic embodiments of the present invention and description thereof are intended to be illustrative of the present invention and do not constitute an undue limitation of the present invention. In the drawings:

FIG. 1 is an exploded schematic diagram of an internal structure of a multifunctional aromatherapy machine according to an embodiment of the present invention;

FIG. 2 is a schematic diagram of an internal structure of a multifunctional aromatherapy machine according to an embodiment of the present invention;

FIG. 3 is a schematic diagram of an overall structure of a multifunctional aromatherapy machine according to the present invention;

FIG. 4 is a circuit block diagram of a control circuit of a multifunctional aromatherapy machine according to an embodiment of the present invention;

FIG. 5 is a power circuit diagram of a control circuit of a multifunctional aromatherapy machine according to an embodiment of the present invention;

FIG. 6 is a boosting circuit diagram of a control circuit of a multifunctional aromatherapy machine according to an embodiment of the present invention;

FIG. 7 is a comparison circuit diagram of a control circuit of a multifunctional aromatherapy machine according to an embodiment of the present invention;

FIG. 8 is a circuit diagram of an MCU main control chip of a control circuit of a multifunctional aromatherapy machine according to an embodiment of the present invention;

FIG. 9 is a fan driving circuit diagram of a control circuit of a multifunctional aromatherapy machine according to an embodiment of the present invention;

FIG. 10 is a key circuit diagram of a control circuit of a multifunctional aromatherapy machine according to an embodiment of the present invention; and

FIG. 11 is a lamplight control circuit diagram of a control circuit of a multifunctional aromatherapy machine according to an embodiment of the present invention.

### DESCRIPTION OF REFERENCE NUMERALS

1—Lampshade; 11—fog outlet hole;

2—water tank; 21—water tank cover; 211—water outlet; 22—water retaining sheet; 23—air outlet; 231—air outlet cover,

3—atomizing sheet; 31—through hole;

4—atomizing sheet pressing plate; 41—first screw;

5—circuit board; 51—second screw;

6—sealing ring;

7—pedestal; 71—aluminum ring, 72—key base; 73—key switch; 74—foglight knob; 75—timing indicator lamp; 721—third screw;

8—Fan; 81—fourth screw;

9—chassis; 91—power interface; 911—fifth screw; 912—power supply socket; 92—gray rubber mat; 921—sixth screw.

### DETAILED DESCRIPTION OF THE EMBODIMENTS

The present invention will be described in detail below with reference to the accompanying drawings and the embodiments. Each example is provided to explain the present invention instead of limiting the present invention. In fact, those skilled in the art will appreciate that modifications and variations may be made in the invention without departing from the scope thereof. For example, features shown or described as one part of one embodiment may be applied to another embodiment to generate yet another embodiment. Therefore, it is expected that the present invention includes such modifications and variations that fall within the scope of the appended claims and their equivalents.

In the description of the present invention, orientation or position relationships indicated by terms “longitudinal”, “transverse”, “upper”, “lower”, “front”, “rear”, “left”, “right”, “vertical”, “horizontal”, “top”, “bottom” and the like are orientation or position relationships shown in the drawings, and these terms are merely for facilitating description of the present invention, but not for requiring that the present invention must be constructed and operated in a specific orientation, and thus, these terms cannot be understood as a limitation to the present invention. As used in the present invention, the terms “connected”, “connection” and “set” should be understood in a broad sense, for example, they may be fixed connection or detachable connection, they may be direct connection or indirect connection through an intermediate part; or they may be wired connection and wireless connection, or may be connection through a wireless communication signal. For those of ordinary skill in the field, the specific meanings of the terms may be understood according to the specific conditions.

The accompanying drawings show one or more examples of the present invention. The detailed description uses reference numerals and letters to refer to the features in the accompanying drawings. Similar numeral references in the drawings and description have been used to refer to the similar parts in the present invention. As used herein, the terms “first”, “second” and “third” are used interchangeably to distinguish one component from another component, and are not intended to indicate the position or importance of individual components.

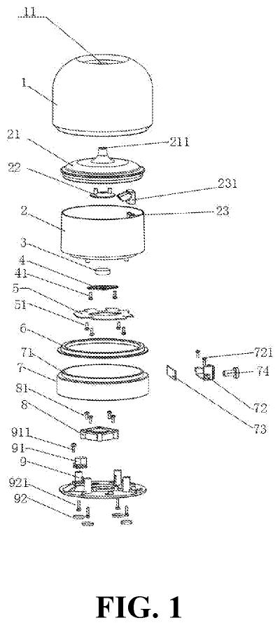

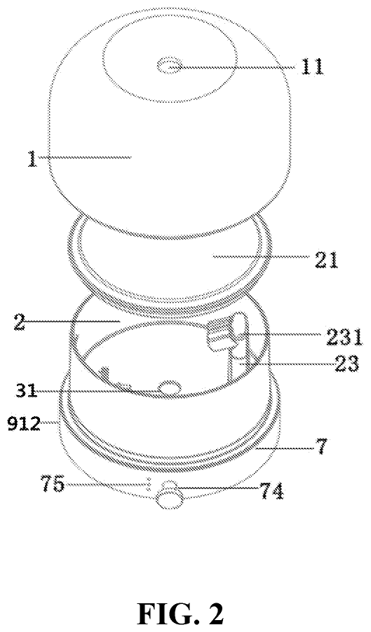

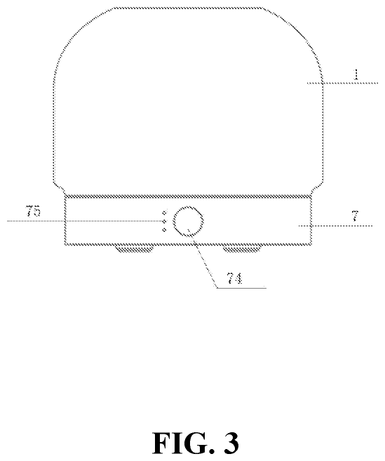

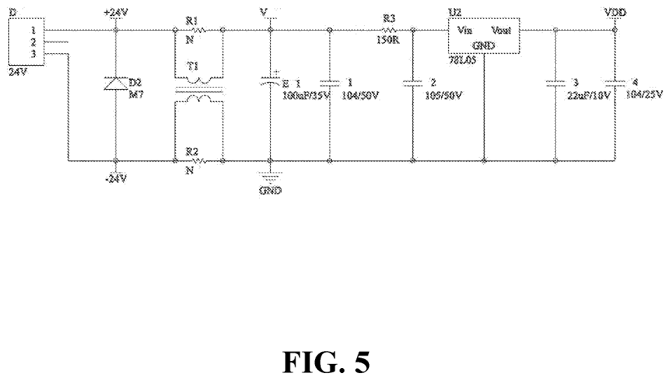

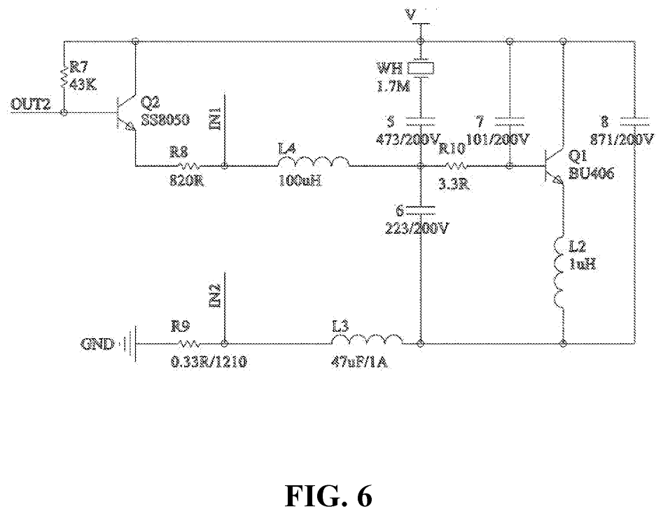

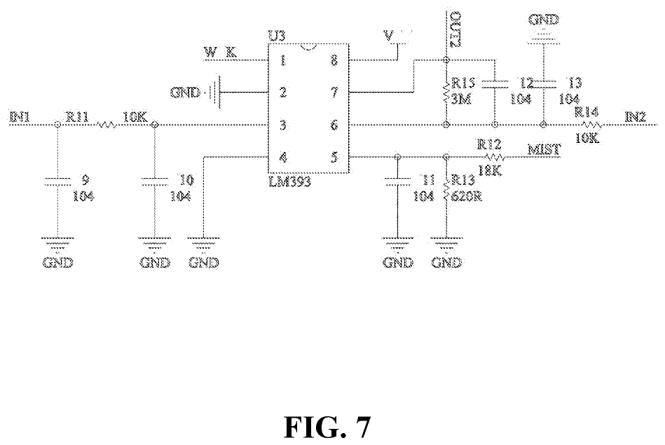

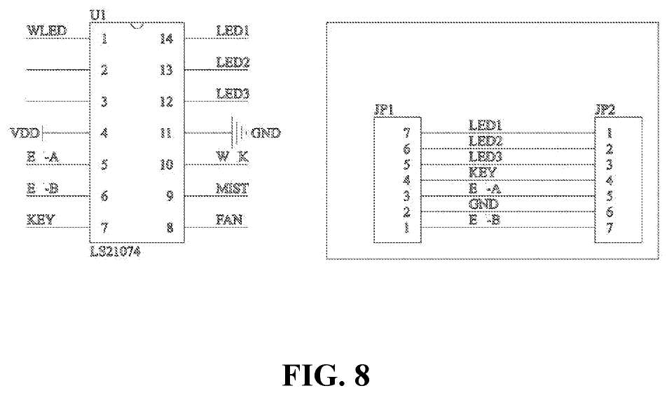

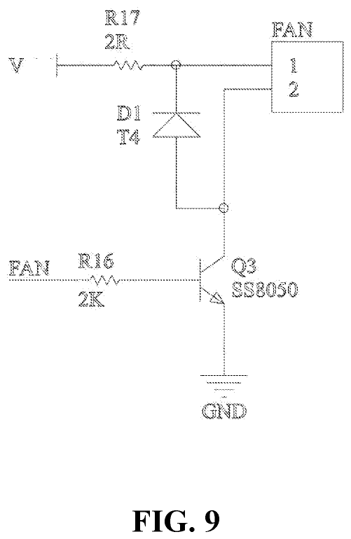

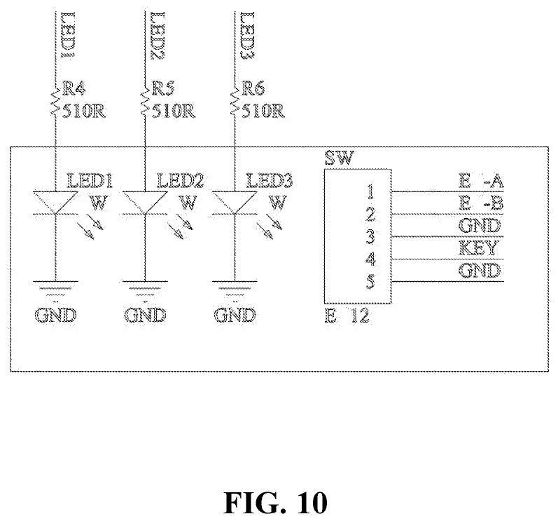

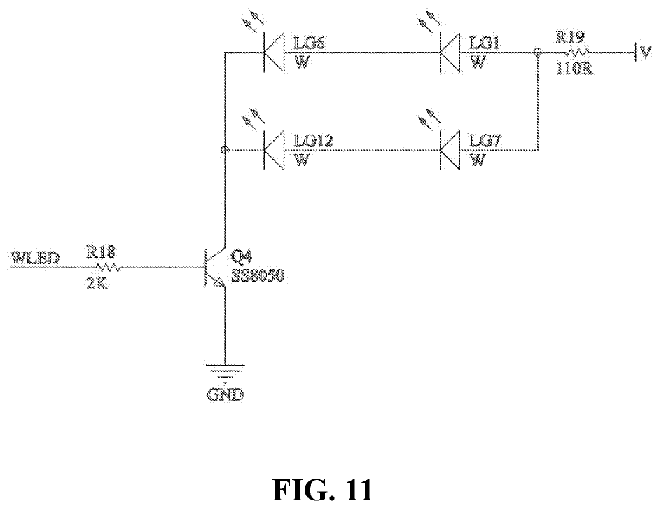

As shown in FIG. 1 to FIG. 11, according to the embodiments of the present invention, a multifunctional aromatherapy machine is provided and includes a pedestal 7, a water tank 2 and a cover body, wherein the water tank 2 includes a through hole 31 formed in a bottom surface of the water tank 2 and communicating with the pedestal; a chassis 9 is arranged below the pedestal 7; the chassis 9 is fixed below the pedestal 7 through a sixth screw 921; the pedestal 7 and the chassis 9 form a built-in space with an opening above; and an anti-skid gray rubber mat 92 adheres to a lower surface of the chassis, so that the aromatherapy machine can be prevented from sliding on a plane. A circuit board 5, an atomizing sheet 3 and an atomizing sheet pressing plate 4 are arranged in the pedestal 7, and the atomizing sheet 3 is fixedly and hermetically connected to the through hole 31 in the bottom surface of the water tank 2 through a first screw 41 and the atomizing sheet pressing plate 4; the water tank 2 further includes an air outlet 23 formed in an inner side wall of the water tank 2 and communicating with the pedestal 7; a height of the air outlet 23 in the water tank 2 is greater than a water surface elevation in the water tank 2, thereby preventing water in the water tank 2 from entering the built-in space of the pedestal 7 from the air outlet 23 because water flowing into the built-in space of the pedestal 7 is prone to damage the circuit board 5 or other electronic parts; a fan 8 for outputting flowing wind to the air outlet 23 is arranged in the pedestal 7; and the fan 8 is used to blow flowing wind to the air outlet 23 and blow fog in the water tank 2 to the fog outlet 211 of the water tank cover 21. The circuit board 5 is arranged above the fan 8 and is fixedly connected to an outer bottom surface of the water tank 2 through a second screw 51; the fan 8 is fixedly connected to the chassis 9 through a fourth screw 81, the fan 8 blows upwards to accelerate heat dissipation of the circuit board 5, and the fan 8 blow-dries the water stain in the chassis 9 through an air inlet hole formed in the bottom surface of the chassis 9; in addition, a plurality of drain holes are formed in the bottom surface of the chassis 9 and are used to drain the water in the pedestal 7.

A power interface 91, a timing indicator lamp 75 and a foglight knob 74 are arranged on an outer side wall of the pedestal 7 respectively, the power interface 91, the timing indicator lamp 75, the key switch 73 and the foglight knob 74 are electrically connected to the circuit board 5 respectively; a key base 72 is arranged on an inner side wall of the pedestal 7, and is fixed on the inner side wall of the pedestal 7 through a third screw 721; one end of the key base 72 is in inserted connection with the foglight knob 74, and the other end of the key base 72 is electrically connected to a key switch 73; and the key switch 73 is electrically connected to the circuit board 5.

The power interface 91 is fixedly on the bottom surface of the chassis 9 through a fifth screw 911, a power supply socket 912 is arranged on the outer side wall of the pedestal 7, the power supply socket 912 is electrically connected to the power interface 91 formed in the bottom surface of the chassis 9, an aluminum ring 71 is arranged on an inner edge of the opening above the pedestal 7, and the water tank 2 is fixedly connected to the pedestal 7 through the aluminum ring 71; and after the aluminum ring 71 is mounted, the opening edge of the pedestal 7 is divided into the aluminum ring 71 and the outer edge of the pedestal 7 where a water channel is mounted. A sealing ring 6 is arranged between the water tank 2 and the pedestal 7, and the sealing ring 6 sleeves the aluminum ring 71 and the upper surface of the outer edge of the pedestal 7. The sealing ring 6 is used to seal the pedestal 7 and the water tank 2 to prevent water from overflowing into the pedestal when water is added, so that the pedestal 7 keeps dry. The cover body includes a water tank cover 21 covering the opening above the water tank 2, a lampshade 1 covering the outer edge of the opening above the pedestal 7, and an air outlet cover 231 arranged in the water tank 2 and covering the air outlet. The water cover tank 21 is provided with a fog outlet 211 communicating with an interior of the water tank 2, a water retaining sheet 22 is arranged at a port of the fog outlet 211 communicating with the interior of the water tank 2, and the water retaining sheet 22 is fixedly connected to a surface of an inner side of the water tank cover 21. The water retaining sheet 22 is provided to prevent water from flowing into the water tank cover 21; and a fog outlet hole 11 is formed at the top of the lampshade 1, and the fog outlet hole 11 of the lampshade 1 corresponds to the size and position of the fog outlet 211 of the water tank cover 21.

The circuit board 5 includes: a control circuit, the control circuit including a power circuit, the power circuit being configured to control or transmit electric energy; a boosting circuit, the boosting circuit being configured to boost a power signal; a comparison circuit, the comparison circuit being configured to convert an analog signal into a digital signal; an MCU main control module, the MCU main control module being configured to control processing circuit information; a key circuit, the key circuit being configured to control a key switch; a lamplight control circuit, the lamplight control circuit being configured to control lamplight intensity or color, an atomizing control circuit, the atomizing control circuit being configured to control the atomizing sheet to perform atomization; and a fan driving circuit, the fan driving circuit being configured to drive the fan to rotate. The power circuit inputs the power signal to MCU main control module through the boosting circuit and the comparison circuit, the MCU main control module controls the lamplight control circuit and the atomization control circuit respectively through key circuit, and the MCU main control module controls the fan driving circuit. The MCU main control module includes a water shortage detection circuit, and the water shortage detection circuit is configured to detect the water amount of the water tank. The atomization control circuit includes a fog out detection circuit, and the fog out detection circuit is configured to detect the atomization amount. The atomization control circuit includes a timing circuit, and the timing circuit is configured to control the fog out time regularly. The lamplight control circuit includes an LED foglight, and the LED foglight includes a sleeping lamp and an atmosphere lamp. The key circuit includes a gear control circuit, and the gear control circuit includes a non-intermittent fog outlet mode gear control circuit and an intermittent fog outlet mode gear control circuit.

### Embodiment 1

As shown in FIG. 1 to FIG. 3, the present invention discloses a multifunctional aromatherapy machine, including a shell, wherein the shell includes a water tank 2 and a pedestal 7; the water tank 2 is arranged above the pedestal 7, an aluminum ring 71 is arranged between the water tank 2 and the pedestal 7, and the water tank 2 is connected to the pedestal through the aluminum ring 71; a through hole 31 is formed at the bottom of the water tank 2, and the through hole 31 is formed at the middle position of the bottom of the water tank 2; the through hole 31 communicates with the pedestal 7; the pedestal 7 includes a circuit board 5, an atomizing sheet pressing plate 4 is arranged on an upper surface of the circuit board 5, the atomizing sheet pressing plate 4 is provided with an atomizing sheet 3, and the atomizing sheet 3 is hermetically and fixedly connected to the through hole 31; the circuit board 5 includes a control circuit; a key base 72 is arranged on an inner surface of the pedestal 7, a key switch 73 is arranged on the key base 72, a foglight knob 74 is arranged on an inner surface and an outer surface of a side wall of the pedestal 7, and the foglight knob 74 controls the key switch 73; a chassis 9 is arranged on a lower part of the pedestal 7, a fan 8 is arranged on the chassis 9, and the fan 8 is fixedly connected to the chassis 9; and the chassis 9 further includes a power interface 91.

In this embodiment, a key base 72 is arranged on the inner surface of the side wall of the pedestal 7, the key base 72 is fixedly connected to the inner surface of the side wall of the pedestal 7, a key switch 73 is arranged on the key base 72, and the key switch 72 is connected to the key base 72 through a third screw 721; a foglight knob 74 is arranged on the outer surface of the side wall of the pedestal 7 corresponding to the key base 72, and the foglight knob 74 controls the key switch 73; a chassis 9 is arranged on a lower part of the pedestal 7, a fan 8 is arranged on the chassis 9, and the fan 8 and the chassis 9 are connected through a fourth screw 81; the chassis 9 further includes a power interface 91; and the power interface 91 is connected to the chassis 9 through a fifth screw 911, and the power interface 91 may be externally connected to a direct current power supply.

In this embodiment, preferably, an water outlet 23 is formed in the water tank, a water retaining sheet 22 is sleeved on the air outlet 23, and an air outlet cover 231 is arranged on the water retaining sheet 22; preferably, the air outlet 23 is formed on one side of the bottom of the water tank, the water retaining sheet 22 is an L-shaped water retaining sheet 22, one end of the water retaining sheet 22 is sleeved on the air outlet 23, and an air outlet cover 231 is arranged at the other end of the water retaining sheet 22. The water retaining sheet 22 is used to prevent water drops generated during atomization from entering the pedestal 7 so as to avoid damage to the circuit board 5, and is additionally used to prevent water from entering the pedestal 7 when water is added.

In this embodiment, the air outlet 23 is used to blow out atomized water drops and the fragrance of the added essence, so that a better atomization effect is achieved.

In this embodiment, preferably, the water tank 2 is cylindrical, the pedestal 7 is also cylindrical, and a size of the water tank 2 corresponds to that of the pedestal 7; a shape and a size of the aluminum ring 71 above the pedestal 7 correspond to those of the pedestal 7; preferably, a water tank cover 21 is arranged at the top of the water tank 2, and a fog outlet 211 is formed at the top of the water tank cover 21; and a shape and a size of the cover body below the water tank cover 21 correspond to those of the water tank 2.

In this embodiment, preferably, the shell includes a lampshade 1, a fog outlet hole 11 is formed at the top of the lampshade 1, and the lampshade 1 covers the pedestal 7. Further, the lampshade 1 is a glass lampshade; and a shape and a size of the lampshade 1 correspond to those of the water tank 2, the lampshade 1 is slightly larger than the water tank cover 21, and the lampshade 1 may conveniently cover the pedestal 7.

In this embodiment, preferably, a sealing ring 6 is arranged between the water tank 2 and the aluminum ring 71. Due to the sealing ring 6, the aluminum ring 71 and the water tank 2 are closer to each other, thereby preventing water in the water tank 2 from leaking to the pedestal 7 and being safe and reliable to use.

In this embodiment, preferably, a power supply socket 912 and a timing indicator lamp 75 are arranged on the outer surface of the side wall of the pedestal 7.

In this embodiment, preferably, a gray rubber mat 92 is arranged at the bottom of the chassis 9, a plurality of gray rubber mats 92 may be provided, and the gray rubber mat 92 is connected to the chassis 9 through a sixth screw 921. Through the gray rubber mat 92, the multifunctional aromatherapy machine is more stable and safer.

### Embodiment 2

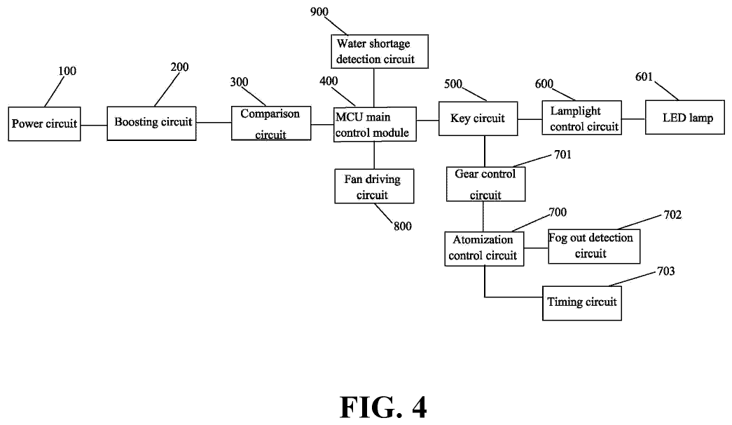

As shown in FIG. 4-FIG. 11, in this embodiment, different from the above embodiment, the control circuit includes: a power circuit 100, the power circuit being configured to provide a power supply;

a boosting circuit 200, the boosting circuit being configured to boost a power signal;

a comparison circuit 300, the comparison circuit being configured to convert an analog signal into a digital signal;

an MCU main control module 400, the MCU main control module being configured to control and process circuit information;

a key circuit 500, the key circuit being configured to control the key switch;

a lamplight control circuit 600, the lamplight control circuit being configured to control lamplight intensity or color,

an atomization control circuit 700, the atomization control circuit being configured to control the atomizing sheet to perform atomization; and

a fan driving circuit 800, the fan driving circuit being configured to drive the fan to rotate,

wherein the power circuit 100 inputs the power signal to MCU main control module 400 through the boosting circuit 200 and the comparison circuit 300, the MCU main control module 400 controls the lamplight control circuit 600 and the atomization control circuit 700 respectively through the key circuit 500, and the MCU main control module 400 controls the fan driving circuit 800.

In this embodiment, preferably, the MCU main control module 400 is a single chip with the model of M68HC11 or M68HC12.

In this embodiment, the MCU main control module 400 may control a lamplight control circuit 600 through a key circuit 500, and the lamplight control circuit 600 may control lamplight intensity or color, so that the multifunctional aromatherapy machine makes people feel comfortable during use; and since the lamplight color can be adjusted, the multifunctional aromatherapy machine is helpful to sleep.

In this embodiment, preferably, the lamplight control circuit 600 includes an LED lamp 601, the LED lamp 601 may be arranged on the side of the pedestal 7, and the LED lamp 601 may also serve as an indicator lamp.

The MCU main control module 400 may also control an atomization control circuit 700 through the key circuit 500, and the atomization control circuit 700 controls the atomizing sheet 3 in the water tank 2 to atomize water, that is, the key circuit 500 may control atomization.

In this embodiment, preferably, the atomization control circuit 700 includes a gear control circuit 701, and the gear control circuit 701 controls the atomization effect through a plurality of gears.

In this embodiment, preferably, the present invention has a two-gear control circuit to control switching of fog outlet modes.

In this embodiment, further preferably, the two-gear control circuit includes a normal fog outlet mode gear control circuit and an intermittent fog outlet mode gear control circuit.

The normal fog outlet mode gear control circuit and the intermittent fog outlet mode gear control circuit include: in an intermittent fog outlet state, the fog outlet state is continuously circulated in a mode of opening for 30 seconds and closing for 30 seconds. After the fog outlet button is longly pressed for 2 seconds, the indicator lamp flashes, and the fog outlet mode is switched successfully at this time. You can adjust the fragrance concentration and the use time of the aromatherapy machine at will by switching different fog outlet modes.

In this embodiment, the fog outlet modes may be switched in any timing state, without affecting the original set timing function.

The multifunctional aromatherapy machine may adjust the lamplight intensity and color by the key circuit 500 through one-key control, may control atomization through one key, is more convenient to use and has more diverse functions.

In this embodiment, preferably, the foglight knob 74 may rotate the button clockwise to turn on the light and increase the brightness, and may rotate the button anticlockwise to reduce the brightness and turn off the light.

In this embodiment, the lamplight control circuit 600 is controlled by the key circuit 500; the lamplight control circuit 600 controls the LED lamp 601; and when the LED lamp 601 serves as a sleeping lamp and after the function of the sleeping lamp is turned off, the warm yellow light will be turned off after 30 minutes. To activate the function of the sleeping lamp, the light button is longly pressed for 2 seconds, three indicator lamps 75 will be turned on sequentially and then be turned off, and the function of the sleeping lamp is activated at this time. The function of the sleeping lamp may be activated only in the full-light and half-light modes. To exit the sleeping lamp mode, the atmosphere lamp switch is pressed shortly. At this time, the atmosphere lamp will be directly turned off.

In this embodiment, preferably, the MCU main control module 400 includes a water shortage detection circuit 900, and the water shortage detection circuit 900 is configured to detect the water amount of the water tank 2.

In this embodiment, the MCU main control module 400 controls the water shortage detection circuit 900 to detect whether the water tank 2 is short of water. Once the water tank 2 is short of water, the MCU main control module 400 controls the key circuit 500 to turn off atomization, thereby protecting the present invention. At the same time, the MCU main control module 400 controls the key circuit 500 to control the LED lamp 601 to flash continuously, thereby achieving an alarming purpose.

According to the present invention, when the functions of “countdown” and “shutdown when no water” take effect, the lamplight will not be turned off by default when being turned on. If you need to turn off the light and fog out at the same time, please follow the following steps.

Cancel the independent light mode: the knob is pressed for 6 seconds, and “3H” and “8H” indicator lamps will flash alternately three times after the three indicator lamps flash at the same time. At this time, the independent lamplight function has been canceled.

Activate the independent light mode: the knob is pressed for 6 seconds, and “1H” and “3H” indicator lamps 75 will flash alternately three times after the three indicator lamps 75 flash at the same time; and at this time, the independent lamplight function has been activated.

In this embodiment, preferably, the atomization control circuit 700 includes a fog out detection circuit 702, and the fog out detection circuit 702 is configured to detect the atomization amount 701.

The MCU main control module 400 controls the fog out detection circuit 702 by the atomization control circuit 700 to detect the atomization amount of the multifunctional aromatherapy machine. When the atomization amount of the multifunctional aromatherapy machine is less than a certain amount, the MCU main control module 400 controls the LED lamp 601 to flash continuously, so that an alarm purpose is achieved, a user may detect the reason of less fog, the multifunctional aromatherapy machine is simpler and more convenient to maintain, and the service life of the multifunctional aromatherapy machine is prolonged.

In this embodiment, preferably, the atomization control circuit 700 includes a timing circuit 703, and the timing circuit 703 is configured to control the fog out regularly.

In this embodiment, the atomization control circuit 700 may output fog regularly by controlling the timing circuit 703, so that the fog out mode is controlled more flexibly and conveniently. In particular, the atomization control circuit 700 is controlled to output fog regularly through the key circuit 500, so that a plurality of functions can be achieved through one-key control and use is greatly facilitated.

The multifunctional aromatherapy machine has exquisite design, high intelligent degree and diversified functions, may achieve various use purposes through one-key control, is more convenient to use and is deeply loved by customers.

The foregoing is merely illustrative of the preferred examples of the present invention and is not intended to limit the present invention, and various changes and modifications may be made by those skilled in the art. Any modifications, equivalent substitutions, improvements, etc. made within the spirit and scope of the present invention should be included within the protection scope of the present invention.

What is claimed is:

1. A multifunctional aromatherapy machine, comprising a pedestal, a water tank and a cover body, wherein the water tank comprises a through hole formed in a bottom surface of the water tank and communicating with the pedestal, and an air outlet formed in an inner side wall of the water tank and communicating with the pedestal;
a chassis is fixedly connected below the pedestal, the pedestal and the chassis form a built-in space with an opening above, and a fan for outputting flowing wind to the air outlet is arranged in the pedestal; a circuit board, an atomizing sheet and an atomizing sheet pressing plate are arranged in the pedestal, and the atomizing sheet is hermetically and fixedly connected to the through hole formed in the bottom surface of the water tank through the atomizing sheet pressing plate; the circuit board is arranged above the fan and is fixedly connected to the outer bottom surface of the water tank; a power interface, a timing indicator lamp, a key switch and a foglight knob are arranged on an outer side wall of the pedestal respectively; the power interface, the timing indicator lamp and the key switch are electrically connected to the circuit board respectively; an aluminum ring is arranged on an inner edge of an opening above the pedestal, and the water tank is fixedly connected to the pedestal through the aluminum ring; and
the cover body comprises a water tank cover covering an opening above the water tank, a lampshade covering an outer edge of the opening above the pedestal, and an air outlet cover arranged in the water tank and covering the air outlet.

2. The multifunctional aromatherapy machine according to claim 1, wherein the circuit board comprises: a control circuit, the control circuit comprising a power circuit and the power circuit being configured to control or transmit electric energy;
a boosting circuit, the boosting circuit being configured to boost a power signal;
a comparison circuit, the comparison circuit being configured to convert an analog signal into a digital signal;
an MCU main control module, the MCU main control module being configured to control and process circuit information;
a key circuit, the key circuit being configured to control the key switch;
a lamplight control circuit, the lamplight control circuit being configured to control lamplight intensity or color,
an atomization control circuit, the atomization control circuit being configured to control the atomizing sheet to perform atomization; and
a fan driving circuit, the fan driving circuit being configured to drive the fan to rotate,
the power circuit inputting the power signal to the MCU main control module through the boosting circuit and the comparison circuit, the MCU main control module controlling the lamplight control circuit and the atomization control circuit respectively through the key circuit, and the MCU main control module controlling the fan driving circuit.

3. The multifunctional aromatherapy machine according to claim 2, wherein the water tank cover is provided with a fog outlet communicating with an interior of the water tank, a water retaining sheet is arranged at a port of the fog outlet communicating with the interior of the water tank, and the water retaining sheet is fixedly connected to an inside surface of the water tank cover.

4. The multifunctional aromatherapy machine according to claim 3, wherein a fog outlet hole is formed at the top of the lampshade, and a size and a position the fog outlet hole of the lampshade correspond to those of the fog outlet of the water tank cover.

5. The multifunctional aromatherapy machine according to claim 1, wherein a sealing ring is arranged between the water tank and the pedestal, and the sealing ring is sleeved on the aluminum ring and an upper surface of an outer edge of the pedestal.

6. The multifunctional aromatherapy machine according to claim 2, wherein the MCU main control module comprises a water shortage detection circuit, and the water shortage detection circuit is configured to detect the water amount of the water tank.

7. The multifunctional aromatherapy machine according to claim 6, wherein the atomization control circuit comprises a fog out detection circuit, and the fog out detection circuit is configured to detect the atomization amount.

8. The multifunctional aromatherapy machine according to claim 7, wherein the atomization control circuit comprises a timing circuit, and the timing circuit is configured to control the fog out time regularly.

9. The multifunctional aromatherapy machine according to claim 8, wherein the lamplight control circuit comprises an LED foglight, and the LED foglight comprises a sleeping lamp and an atmosphere lamp.

10. The multifunctional aromatherapy machine according to claim 2, wherein the key circuit comprises a gear control circuit, and the gear control circuit comprises a non-intermittent fog outlet mode gear control circuit and an intermittent fog outlet mode gear control circuit.

11. The multifunctional aromatherapy machine according to claim 1, wherein a key base is arranged in a side wall of the pedestal, the foglight knob is inserted into the key base, and the key base is electrically connected to the key switch.

12. The multifunctional aromatherapy machine according to claim 1, wherein a lower surface of the chassis adheres to an anti-skid gray rubber mat.
